(19) United States
(12) Patent Application Publication
(10) Pub. No.: US 2023/0173167 A1
(43) Pub. Date: Jun. 8, 2023

(54) AUTOMATED DRUG MICROINJECTION PUMP WITH WIRELESS COMMUNICATION AND METHOD OF USE

(71) Applicant: POCKET CLINIC TECNOLOGIA DA INFORMAÇÂO LTDA,, Säo Paulo-SP (BR)

(72) Inventors: Amir Hossein Omidvar, North York (CA); Atena Amanati Shahri, North York (CA); Robert John Horacek, Park City, UT (US); Raymond Alfred Horacek, Yokohama (JP)

(21) Appl. No.: 17/924,236

(22) PCT Filed: May 12, 2020
(86) PCT No.: PCT/BR2020/050155

§ 371 Date: Nov. 9, 2022

(51) Int. Cl. A61M 5/142; A61M 5/172; A61M 5/158

(52) U.S. Cl. CPC A61M 5/14224; A61M 5/172; A61M 5/158; A61M 2205/0238

(57) ABSTRACT

The invention describes an automated drug microinjection pump with wireless communication and method of use, which is a device that aims to help chronic patients live healthy lives without the constant burden of difficult treatment. It is designed to present personalized, pain-free, simple and cost-effective treatments for chronic patients who constantly need medicines. This device continuously adapts to the developments in chronic patients' lives without the need for constant attention. This solution optimizes the drug treatment based on the patient's personal medication dosing ratios which are measured, assessed and optimized using artificial intelligence.

The invention is an automated drug microinjection pump with wireless communication and method of use, is a device that aims to help chronic patients live healthy lives without the constant burden of difficult treatment. It is designed to present personalized, pain-free, simple, and cost-effective treatments for chronic patients who constantly need medicines. This device continuously adapts to the developments in chronic patients' lives without the need for constant attention. This solution optimizes the drug treatment based on the patient's personal medication dosing ratios which are measured, assessed, and optimized using artificial intelligence.

### APPLICATION FIELD

The invention is about to produce the smallest and easy to use drug delivery system that has the ability to communicate wirelessly. It is destined to help chronic patients live healthy lives without the constant burden of difficult treatment and It is designed to present personalized, pain-free, simple, and cost-effective treatments for chronic patients who constantly need medicines.

### INVENTION PROPOSAL

The object of the present invention is to provide a wearable drug infusion pump that is controlled by a daily use gadget like a smartphone or smartwatch which connects wireless techniques such as Bluetooth or IEEE 802.15.4 wireless standards such as ZigBee 3.0 and Open thread. The device attaches to the patient's body with adhesive and infuses the desired amount of medication via a medical-grade stainless steel or silicone cannula. The medication dosage is calculated by the software embedded in the gadget. The device has the ability to run on a battery that is rechargeable and has the capability of charging wirelessly or wired.

### PRIOR ART

The state of the art in infusion pumps presents several approaches. The available pumps in the market are normally composed of a piston or piezoelectric infusion mechanisms. In the current pumps, the container in the pump holds the medicine which will be injected into the human body through the tubing. Some of them control the pump functions remotely by using a PDA, and in the other displays and buttons are embedded on the pump body. Disposable patches attached to the body are used in some pumps however most of the infusion pumps are durable devices. A patent search was performed in the WIPO repository and they are listed below with a brief description of its operation. Below are the most relevant:

The patent document WO2010078508, INFUSION PUMP SYSTEM AND METHODS, this invention describes, some embodiments an infusion pump system can provide a suggested bolus dosage based on particular parameters (e.g., the user's recent blood glucose characteristics, food intake data, an amount of insulin already delivered to the user which has not yet acted on the user, or other factors). In some circumstances, the infusion pump system can receive information indicative of the user's blood glucose level and suggest an insulin bolus dosage that is at least partially dependent upon the recent rate of change in the user's blood glucose level. Such a feature can be helpful to a user when the infusion pump is operated in conjunction with a glucose monitoring device because the suggested bolus dosage can be at least partially based on recent data indicative of the user's blood glucose level;

The patent document WO2005120606, DRUG DELIVERY SYSTEM, this invention describes; a drug delivery system (10) has a positive pressure drug reservoir (15). A pump (19) is in fluid communication with the reservoir (15) via a first fluid passageway (18). The outlet (19b) of the pump (19) is in fluid communication with a first regulator (21). The regulator (21) includes a housing (24) that has a first subchamber (24a) and a second subchamber (24b). The second subchamber (24b) is divided into a lower chamber (28a) and a middle chamber (28b) by a diaphragm (28). A third fluid passageway (32) is positioned between the lower chamber (28a) and the drug reservoir (15), whereby accidental flow from the reservoir (15) to an outlet (21b) is prevented;

The patent document JP2003126250, INSULIN-INJECTING DEVICE, this invention describes a device to detect and remove the clogging of a channel to feed insulin at an early stage from a reservoir of the insulin in an intracorporeal implanted type insulin-injecting device. Where, a flow meter 16 is provided in a catheter 12, which feeds insulin from the insulin reservoir 10. When the insulin flow rate of the catheter 12 becomes a specified value or lower, the generation of clogging due to the deposit and the adhesion of the insulin on the internal wall of the catheter 12 is judged. When the clogging is detected, the catheter is pressed by piezoelectric elements 20 and 22 which are provided on the catheter 12, and the internal pressure of the catheter 12 is increased. By the pressure-volume, the clogging is pushed out from the distal end of the catheter, and the clogging is removed;

The patent document JP2002126092 (U.S. Pat. No. 6,589,229), WEARABLE, SELF-CONTAINED DRUG INFUSION DEVICE, this invention describes a device to provide a portable drug infusion device which can accurately control a required critical dosage of a drug like insulin. Where a piezoelectric valve or a pump structure is used by combining a thermal flow sensor and a closed-loop control circuit that controls a required flow. The drug infusion device 30 is made in extremely small dimensions and closely controls the flow of insulin and other drugs. This device is structurally divided into two sections, namely a section for recycling expensive electronic components 34 and a section for disposable liquid delivery components that can be separated 32, so that the device can be reduced cost, a drug can be thoroughly sterilized and the contamination of recycling liquids can be prevented by limiting the flow routes of disposable liquids. Thus, it is possible to electronically control the variation of drug flows by changing the flow and/or a flow programming, thereby conforming to the variety of requirements of a patient, time, and delivery rate. The present invention differs from this document mainly in that: the way of injecting the medicine is performed through a needle powered by a programmed actuator and the dose of medicine is calculated and activated through a diaphragm micropump driven by mechanical elements such as springs, which reduces its size, leaving the device more comfortable for the user;

The patent document GB2525149, THERAPEUTIC PRODUCT DELIVERY DEVICE, this invention describes a therapeutic product delivery device which comprises a device body 21 and a cartridge 26 for holding a therapeutic product. An engagement structure 410, 420 is provided for releasably engaging the cartridge 26 with the device body 21. A fault detector is provided for detecting a fault in the delivery of the therapeutic product from the cartridge. A release trigger 430, 440 is responsive to the detection of a fault to cause the engagement structure 410, 420 to release the cartridge 26 from the device body 21. In this way, a fault causes the cartridge to be ejected from the device body 21, which will prevent any further delivery of the therapeutic product to the patient. The cartridge 26 may also be manually detached using release actuator 460. The therapeutic device may be an insulin pump device;

The document CN204379897, INFUSION PUMP, this invention describes, the utility model that discloses an infusion pump which comprises a casing, and a power supply, a motor, a driving component and a control component which are arranged in the casing, as well as a medicine storage component, wherein at least part of the medicine storage component is accommodated in the casing; the medicine storage component is provided with an infusion tube and an infusion needle for outputting liquid medicine; the motor drives the medicine storage component to output liquid medicine from the infusion needle through the driving component under the control of the control component; the infusion pump further comprises an adhering component arranged on the casing and is adhered on the skin near an area to be injected of a patient by the adhering component. The infusion pump is provided with the short infusion tube which is directly adhered on a human body to inject insulin for the patient so that the infusion pump is small in volume, light in weight and convenient to carry; moreover, the freedom of movement of a wearer can be ensured, and the use cost is reduced;

The patent document EP2877945 (WO2014016230), SYSTEM FOR MANAGING INSULIN DOSAGE DATA, this invention describes, a method that provided for managing insulin dosage data collected over a complete day and a partial day. The method includes providing, by an insulin pump, a plurality of insulin dosages over a complete day and a partial day and recording the insulin dosages. The method also includes recognizing completion of the complete day, recording an end-of-day marker, and associating the end-of-day marker to the dosages delivered during the complete day. Moreover, the method includes identifying complete day dosage data and partial day dosage data. Additionally, the method includes performing a calculation of insulin dosage statistics and/or displaying and distinguishing between the complete day dosage data and the partial day dosage data. The calculation includes the complete day dosage data and excludes the partial day dosage data;

The patent document WO2012134589, SELF-CONTAINED TORSION SPRING INSERTER FOR DRUG DELIVERY INFUSION SET, this invention describes, an infusion set (201) including a base (221) and a flexible catheter (242) movable from a first catheter position disposed substantially entirely within the base (221) to a second catheter position in which a free end of the catheter (242) is disposed externally of the base (221). An introducer needle (234) is located within the catheter (242) and is movable between a first introducer needle position disposed substantially entirely within the base (221) and a second introducer needle position in which a free end of the introducer needle (234) is disposed externally of the base (221). A torsion spring (281) is activated to move the catheter (242) from the first to the second catheter position and the introducer needle (234) from the first to the second introducer needle position to facilitate insertion of the catheter (242). The introducer needle (234) is thereafter moved by the torsion spring (281) back to the first introducer needle position to store the introducer needle (234) within the base (221) with the free end of the catheter (242) remaining disposed externally of the base (221). The present invention differs from this document mainly in that: the way of injecting the medicine is performed through a needle powered by a programmed actuator without catheters and the dose of medicine is calculated and activated through a diaphragm micropump driven by mechanical elements such as springs, and because it has a disposable part;

The patent document US20140194819, MEDICATION INFUSION SET, this invention describes, an infusion set that is provided for use in delivering fluid through a cannula, which is housed on a cannula housing, to a selected subcutaneous infusion site on a patient. The fluid is generally a medication, e.g., insulin. The cannula is in fluid communication with a fluid passageway surrounded by a projection on the cannula housing that includes one or more rail-like extensions acting as guides. A connector connects the cannula housing to a fluid delivery system, e.g., an infusion pump. The connector includes a needle, one or more guide arms that slide over the rail-like extensions to guide the needle into the self-sealing septum, and one or more locking arms, with barbs at the end, to connect with corresponding recesses in the cannula housing. An introducer having one or more similar guide arms and locking arms, and a longer needle, may be used to insert the cannula into the patient; and

The patent document WO2004020020, ADAPTER CONNECTOR FOR AN INFUSION SET AND INSERTER SYSTEM, this invention describes, an adapter is provided for use with an infusion set and inserter device used for placement of a subcutaneous infusion set or the like used for delivering a selected medication to a patient. The adapter comprises a first end adapted to be removable coupled to the infusion set and a second end adapted to be removable coupled to an insertion device, particularly the sled fixtures in a track of the insertion tool. The adapter is designed for receiving and supporting an infusion set in a position in the inserter which is used for placement of insertion of the needle or cannula through the skin of a patient at a selected insertion site. The provided adapter connectors enable the use of a variety of insertion sets having different configurations with a given inserter having a given configuration; and

The patent document BR 10 2017 026112 3, DRUG INFUSION PUMP WITH DISPOSABLE CARTRIDGE CONTROLLED BY CELLULAR. It consists of a compact medication infusion pump (1) for the treatment of chronic diseases such as diabetes and cancer, consisting of a controller (2) and a disposable cartridge (3) where a piezoelectric diaphragm micropump (4) and the flask (F) in a commercial standard of medicine to be injected, and the infusion pump (1) has a sensor (5) of malfunction and connects to the patient's body through a cannula and injects the amount of desired medicine according to the commands sent by the application installed on the cell phone/smartphone, which makes all the control/user interface with the equipment via Bluetooth. The present invention differs from this document mainly in that: it does not contain the original medicine bottle and various sized medicine containers from 1 ml to 5 ml has been included in this invention; the way of injecting the medicine is performed through a spring loaded needle that penetrates to skin. The dose of medicine is calculated and infused through a diaphragm micropump driven by mechanical elements such as a programmed actuator, which reduces its size, resulting in a more comfortable device for the user. The different methods of communication have been applied in this invention such as Bluetooth or IEEE 802.15.4 wireless standards such as ZigBee 3.0 and Open thread.

### INVENTION GENERAL DESCRIPTION

The present invention aims to provide a compact, user-friendly solution which is more affordable for the treatment of chronic diseases. The proposed invention uses disposable diaphragm micropump technology. The smart injector is divided into two parts: The main part includes electrical components, electrical boards, battery, diaphragm pump actuator, and their components. The disposable part includes a built-in magnet diaphragm pump cartridge, the drug container which is made of medical-grade silicone, microneedle, and actuator for launching the needle with the mechanism described below.

The drug container has various capacities from 1 ml to 5 ml. In order to make this product waterproof and connect the two parts together, Multiple techniques have been applied that are described below. In addition, we use end to end encryption techniques to secure the communication between the smart injector and the gadget (Smartphone or Smartwatch).

The current solutions are normally big in size and complicated to work with. This invention can totally be controlled by any kind of daily use gadgets like a Smartphone or Smartwatch, contrary to the state of the art using external remote controls or displays and buttons on the infusion pump itself, increasing its dimensions. Using a piston pump makes the device size big and adding the control part like electronics and battery makes it heavy.

### BRIEF DRAWINGS DESCRIPTION

FIG. 1 shows the automated drug microinjection pump this view shows both disposable and reusable portions, the entire assembly with wireless communication in a perspective view showing the external details

FIG. 2 shows the automated drug microinjection pump (reusable main part) with wireless communication in a perspective view showing the main part of internal details.

FIG. 3 show the automated drug microinjection pump disposable with wireless communication in a perspective view showing the main part internal details emphasizing the component and operating elements

FIG. 4 shows the automated drug microinjection pump with wireless communication in a perspective view showing the bottom and/or contact with the patient, highlighting the medicine injection needle

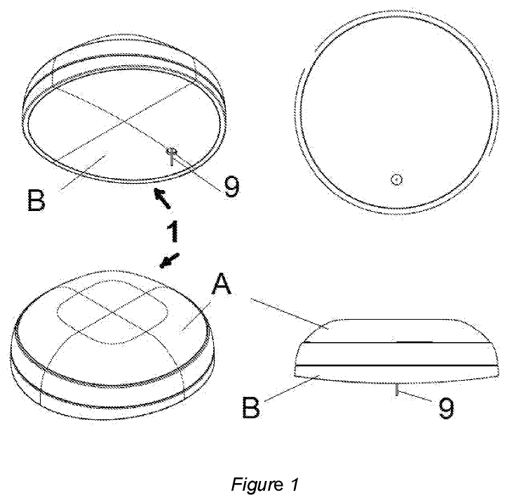

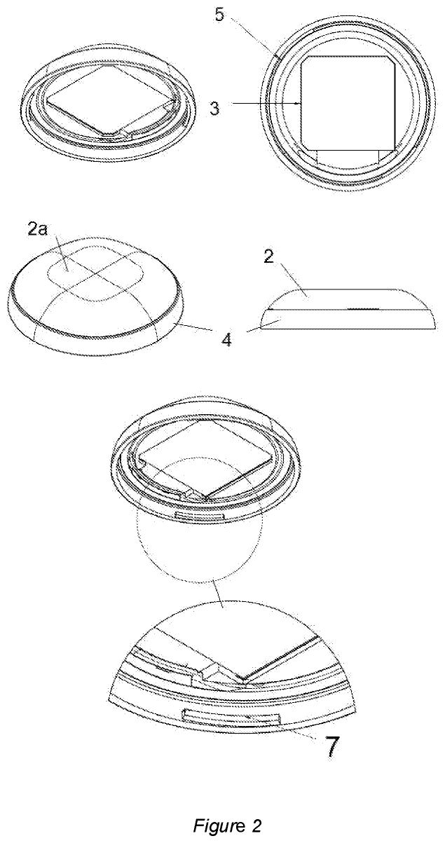

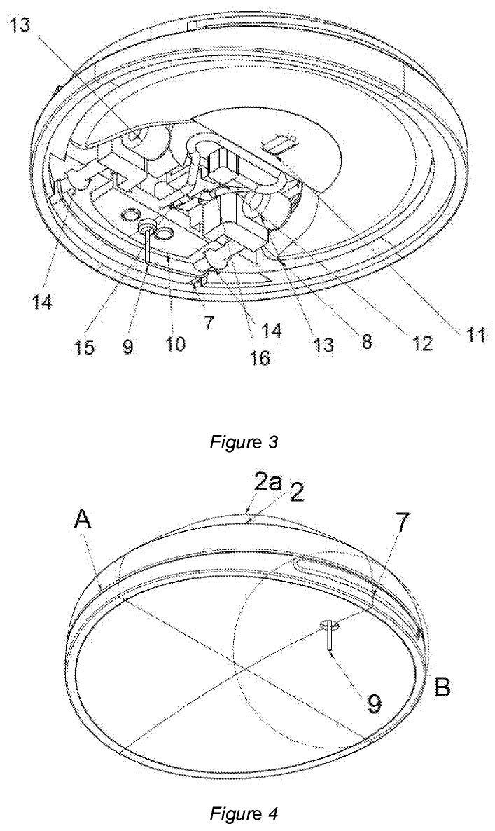

### DETAILED DESCRIPTION OF THE EMBODIMENTS OF THE INVENTION

In the proposed invention, the automated drug microinjection pump with wireless communication is an automatic device (1) where a disposable diaphragm micropump technology has been deployed.

This device is a smart injector. The device has a tablet-shaped housing with perimeter, oval, circular, and or semi-circular, to facilitate handling and use, this housing divided into two parts. The smart injector is divided into two parts: The main part (A) that includes electrical components, electrical boards, battery, and a diaphragm pump actuator (3) and their components.

The main part is a controller and includes two medical-grade plastic casing and one medical-grade metal ring, the electrical components, electrical boards, battery, on-off switch (7), and diaphragm pump actuator (3). This part is autonomous for three days and then is charged wirelessly or wired. This main part also comprises a top part (2) with an outer casing and (2a) and a bottom casing (4) to connect with a disposable part (B) through the main seal ring (5) that provides the connection as well as pressure for waterproofing.

The second part is the disposable part (B) that includes a built-in magnet diaphragm pump cartridge comprises a drug container (1) which is made of medical-grade silicone, micro needle (9) made of medical grade stainless steel or silicone and a support needle hub (16) actuator to launch the needle made of medical-grade plastic, by applying the small force the needle releases because of the pressure of spring. called micro needle (9) connected to the drug container (8) by a customized tube (15), made of medical-grade stainless steel or silicone, and a medical-grade stainless steel tubing, an input tubing (11) and an output tubing (12) which connects the diaphragm pump to the needle hub (10) and a medical-grade silicone drug container (8) with the various capacities from 1 ml to 5 ml. The support needle hub (10) actuator comprises double torsion spring (13) with their respective actuating rods (14) arranged in the spring body (16).

In this invention the medicine contained has been designed in various sizes such as 1 ml, 1.5 ml, 2 ml, 2.5 ml, 3 ml, 3.5 ml, 4 ml, 4.5 ml, 5 ml. Indeed, this invention has the capacity to apply any medicine that needs to be injected continuously to the human body in different volumes and in different dosages.

These two parts (A, B) are attached together with two mechanisms, a built-in diaphragm pump cartridge magnet, using a medical-grade circle or square shape medical-grade steel in the main part (A) and built-in magnet plate has deployed in the disposable part (B) makes parts (A), (B) attach together. Using a built-in magnet and steel plates make two parts (A), (B) indivisible. In order to separate two parts (A), (B), it should apply external force.

A second method that is used in this invention is a metal seal ring that rotates 90 degrees to press and fix these two parts. The main seal ring (5), is used to securely attach the disposable cartridge, by a turning motion using thread: this uses the principle of a ramp to apply large amounts of downward force on the gasket/waterproofing seal, with low user force required (good mechanical advantage). The turning motion is easy, convenient, and intuitive for users of all ages and strength abilities, it is necessary for waterproofing/gasket seal. That action is easy for consumers to change/refill cartridges, just take one cartridge off and put a new one on, no need to mess with tubes, or refill the cartridge. Although it is not restricted to such a mechanism, there are other ways of attaching the cartridge as well.

There are other ways that could be used to generate the closing force to attach the reusable side of the unit with the disposable cartridge—these could be latches, clamps, cam locks, screws, magnets, adhesive, etc. The mentioned features make the invention to detect when the cartridge is connected correctly or not.

This invention has used a built-in rubber gasket (5), in order to make the invention sealed and waterproof. The gasket shape is like a ring and the cross-section shape of a rubber gasket is like a circle, triangle, oval or square. by using a downward force from two previous sealing methods in [027-028], a built-in rubber gasket makes the invention waterproof.

This invention has an easy method to insert the needle into the patient's body. The actuating button and activation method to insert the needle method for the automated drug microinjection pump with wireless communication, the method of using the device (1) comprises: a button (7) with easy push motion, with an ergonomic and easy to do for all ages squeeze/push motion where there is a small force required, as it is a strong position for the hand, and is comfortable for fingers/thumb/user, the motion does not interrupt waterproofing, still is waterproof before and after activation due to two o-rings on the button activation rods (14), having a lock to hold button/needle in place until to release and an audible/visual sign to a user that it activated correctly, or warning if not activated

After activation by the button (7) the needle is spring-loaded so with one motion by the user to press the button the needle will launch automatically.

This invention is a tubeless device and wearable, using the technique to make two separate parts sealed and waterproof and durable.

The automated drug microinjection pump with wireless communication, also comprises the advantages, that to hold: a wireless communication (such as Bluetooth™ and IEEE 802.15.4 wireless standards such as ZigBee 3.0 and Open thread), using an NFC tag to connect wirelessly and more secure when the communication secured with end-to-end encryption; BENEFITS

The main advantages of the invention are: the Invention is wearable and it attaches with adhesive to the patient's body. The pairing with the gadget is via NFC-tag which makes the communication more secure; the next step is to attach the invention to the patient's body and launch the needle; this invention makes it simple by using a small force push to the actuator, consequently, the needle will be released from the housing and will be inserted into the body therefore due to force of the spring at the back of the needle hub; and the main part, the controller, is durable and is not single-used but the cartridge is disposable the lifetime of the cartridge highly depends on the patient's usage which is estimated between 2 or 3 days. The present invention does not contain the original medicine bottle; the way of injecting the medicine is performed through a spring-loaded needle that penetrates to skin. The dose of medicine is calculated and infused through a diaphragm micropump driven by mechanical elements such as a programmed actuator, frequency and voltage, which reduces its size, resulting in a more comfortable device for the user. The seal metal ring and built-in magnet methods for connecting the reusable main part to a disposable cartridge make this invention easy to turn and replace cartridges. This invention uses artificial intelligence and machine learning to track the chronic disease and optimizes the drug treatment based on the patient's personal medication dosing ratios which are measured, assessed, and optimized using artificial intelligence.

1. AUTOMATED DRUG MICROINJECTION PUMP WITH WIRELESS COMMUNICATION AND METHOD OF USE, the automated drug microinjection pump with wireless communication is an automatic device (1) with a disposable diaphragm micropump technology, wherein the device (1) is an automatic smart injector, it has a tablet-shaped housing with perimeter, oval, circular and or semi-circular, this housing divided into two parts: the main part (A) that includes electrical components, electrical boards, battery, and a diaphragm pump actuator (3) and their components and the second part is the disposable part (B), wherein called these two parts (A, B) are attached together with one or more mechanisms, a built-in cartridge magnet and/or a metal ring that rotates 90 degrees to press and fix these two parts, and also, it has a built-in rubber gasket (5), as an o-ring, in order to make the invention sealed and waterproof.

2. AUTOMATED DRUG MICROINJECTION PUMP WITH WIRELESS COMMUNICATION AND METHOD OF USE, the device (1) according to claim 1, wherein the main part (A) is a controller and includes two medical-grade plastic, one medical-grade metal ring, the electrical components, electrical boards (PCBs), battery, on-off switch (7) and diaphragm pump actuator (3); called main part (A) is autonomous for three days and then is charged wirelessly or wired; called main part (A) also comprises a top part (2) with an outer casing (2a) and a bottom casing (4) to connect with a disposable part (B) through a main seal ring (5) that provides the connections as well as pressure for waterproofing.

3. AUTOMATED DRUG MICROINJECTION PUMP WITH WIRELESS COMMUNICATION AND METHOD OF USE, the device (1) according to claim 1, wherein the second part is the disposable part (B) that includes a built-in magnet diaphragm pump cartridge comprises a drug container (8) which is made of medical-grade silicone, micro-needle (9) made of medical grade stainless steel or silicone and a support needle hub (10) made of medical-grade plastic, called micro-needle (9) connected to the drug container (8) by a medicine/drug tube (15), made of medical-grade stainless steel or silicone, and a medical-grade stainless steel tubing, including an input tubing (11) and an output tubing (12) which connects the diaphragm pump to the needle hub (10) and the medical-grade silicone drug container (8); called needle hub support (10) actuator comprises a double side torsion spring (13) with their respective actuating rods (14) located in the spring body (16).

4. AUTOMATED DRUG MICROINJECTION PUMP WITH WIRELESS COMMUNICATION AND METHOD OF USE, the device (1) according to claim 2, wherein the device (1), a circle, oval or square shape steel plate has been deployed in the main part (A); a built-in magnet plate deployed in the disposable part (B); using a built-in magnet and stainless-steel plates make parts (A) and (B) indivisible; in order to separate two parts (A) and (B), an external force should be applied.

5. AUTOMATED DRUG MICROINJECTION PUMP WITH WIRELESS COMMUNICATION AND METHOD OF USE, the device (1) according to claim 2, wherein the main seal ring (5) securely attaches disposable cartridge, an easy turning motion using thread has been deployed; this uses the principle of a ramp to apply large amounts of downward force on the gasket/waterproofing seal, with a small user force required.

6. AUTOMATED DRUG MICROINJECTION PUMP WITH WIRELESS COMMUNICATION AND METHOD OF USE, the device (1) according to claim 1, wherein the device (1) is able to present other ways that could be used to generate the closing force to attach the reusable side of the unit with the disposable cartridge, these could be latches, clamps, cam locks, screws, magnets, adhesive, and others.

7. AUTOMATED DRUG MICROINJECTION PUMP WITH WIRELESS COMMUNICATION AND METHOD OF USE, the device (1) according to claim 1, wherein the device (1) has used a built-in medical grade rubber gasket (5), in order to make the invention sealed and waterproof; the ring shape built-in gasket that has the cross section shape such as circle, triangle, oval or square, has been deployed; By using a downward force from three previous sealing methods according to claims 4, 5, 6, the built-in rubber gasket makes the invention waterproof.

8. AUTOMATED DRUG MICROINJECTION PUMP WITH WIRELESS COMMUNICATION AND METHOD OF USE, the device (1) according to claim 3, wherein the drug container (8) has the capacity of 1 ml.

9. AUTOMATED DRUG MICROINJECTION PUMP WITH WIRELESS COMMUNICATION AND METHOD OF USE, the device (1) according to claim 3, WHEREIN the drug container (8) has the capacity of 1.5 ml.

10. AUTOMATED DRUG MICROINJECTION PUMP WITH WIRELESS COMMUNICATION AND METHOD OF USE, the device (1) according to claim 3, wherein the drug container (8) has the capacity of 2 ml.

11. AUTOMATED DRUG MICROINJECTION PUMP WITH WIRELESS COMMUNICATION AND METHOD OF USE, the device (1) according to claim 3, wherein the drug container (8) has the capacity of 2.5 ml.

12. AUTOMATED DRUG MICROINJECTION PUMP WITH WIRELESS COMMUNICATION AND METHOD OF USE, the device (1) according to claim 3, wherein the drug container (8) has the capacity of 3 ml.

13. AUTOMATED DRUG MICROINJECTION PUMP WITH WIRELESS COMMUNICATION AND METHOD OF USE, the device (1) according to claim 3, wherein the drug container (8) has the capacity of 3.5 ml.

14. AUTOMATED DRUG MICROINJECTION PUMP WITH WIRELESS COMMUNICATION AND METHOD OF USE, the device (1) according to claim 3, wherein the drug container (8) has the capacity of 4 ml.

15. AUTOMATED DRUG MICROINJECTION PUMP WITH WIRELESS COMMUNICATION AND METHOD OF USE, the device (1) according to claim 3, wherein the drug container (8) has the capacity of 4.5 ml.

16. AUTOMATED DRUG MICROINJECTION PUMP WITH WIRELESS COMMUNICATION AND METHOD OF USE, the device (1) according to claim 3, wherein the drug container (8) has the capacity of 5 ml.

17. AUTOMATED DRUG MICROINJECTION PUMP WITH WIRELESS COMMUNICATION AND METHOD OF USE, the device (1) according to claim 1, wherein the device (1) holds: a wireless communication (such as Bluetooth™ and IEEE 802.15.4 wireless standards such as ZigBee 3.0 and Open thread), using an NFC tag to establish wireless communication with the device by simply touching device claim 1.

18. AUTOMATED DRUG MICROINJECTION PUMP WITH WIRELESS COMMUNICATION AND METHOD OF USE, the device (1) according to claim 1, wherein the device (1), for more secure communication an end-to-end encryption method has been applied for transferring data.

19. AUTOMATED DRUG MICROINJECTION PUMP WITH WIRELESS COMMUNICATION AND METHOD OF USE, a method for use the device (1), wherein the actuating button and activation method to insert the needle for the automated drug microinjection pump with wireless communication, comprises: a button (7) with easy push motion, with an ergonomic and easy to do for all ages squeeze/push motion where there is a small force required, as it is a strong position for the hand, and is comfortable for fingers/thumb/user, the motion does not interrupt waterproofing, still is waterproof before and after activation due to two o-rings on the button activation rods (14), having a lock to hold button/needle in place until to release and an audible/visual sign to user that it is activated correctly, or warning if is not activated; after activated by the button (7) the needle is spring loaded with one motion by the user to press the button, the needle will be inserted automatically.
